(19) United States
(12) Patent Application Publication
(10) Pub. No.: US 2025/0099711 A1
(43) Pub. Date: Mar. 27, 2025

(54) METHOD FOR CONTROLLING SYSTEM COMPRISING PORTABLE AUDIO OUTPUT DEVICE AND MOBILE DEVICE

(71) Applicant: LG ELECTRONICS INC., Seoul (KR)

(72) Inventors: Seokho YOON, Seoul (KR); Soonhyun PARK, Seoul (KR); Kiyoung LEE, Seoul (KR)

(73) Assignee: LG ELECTRONICS INC., Seoul (KR)

(21) Appl. No.: 18/727,235

(22) PCT Filed: Jan. 9, 2023
(86) PCT No.: PCT/KR2023/000377

§ 371 Date: Dec. 13, 2024

(30) Foreign Application Priority Data
Jan. 13, 2022 (KR) 10-2022-0005441

(51) Int. Cl. A61M 21/02; A61B 5/374; A61M 21/00

(52) U.S. Cl. CPC A61M 21/02; A61B 5/374; A61M 2021/0027

(57) ABSTRACT

According to an application (related to meditation and downloadable to a mobile device) according to one embodiment of the present invention, after the meditation-related application is executed, the state of a user wearing a portable audio output device connected to the mobile device in a wired or wireless manner is checked; a segment corresponding to a first layer of specific audio content is output through the portable audio output device according to the checked user's state; and if the checked user's state is changed, a segment corresponding to a second layer of the specific audio content is output through the portable audio output device.

### TECHNICAL FIELD

The present disclosure relates to a portable audio output device, a mobile device, etc. More specifically, for example, it is applicable to all technologies that implement meditation-related applications using wearable devices (e.g., earbuds, smart watches, etc.), smartphones, and the like.

### BACKGROUND ART

As the amount of time spent at home increases after COVID-19, user needs for meditation-related services are increasing.

Meditation is the quiet closing of your eyes and not thinking about anything in a calm state. Meditation is often used to cleanse the mind, reduce stress, promote rest, or train the mind.

According to several research studies, for example, based on verifying the wavelength of the brain during meditation, there is an effect on brain and body hormones.

People may gather offline and receive meditation services through leaders, but recently, the number of people who enjoy meditation services individually online is increasing after COVID-19. A typical online system may be said to be a meditation-related application.

However, a meditation-related application according to a related art is limited to an online meditation service that fails to provide feedback to a user at all.

Therefore, meditation-related beginners have a problem of spending a lot of time and money to find meditation, content, and the like that are suitable for them.

### DISCLOSURE

### Technical Tasks

One technical task of the present disclosure is to provide a technology capable of measuring a user's state prior to a full-fledged meditation service.

Another technical task of the present disclosure, even after starting a meditation service (e.g., outputting audio content), is to change a real-time guide depending on a changed state of a user.

Further technical task of the present disclosure is to implement a service that provides specific feedback through comparison with past data and the like even after a meditation service is terminated.

The technical tasks to be achieved in the embodiments are not limited to the above-mentioned matters, and other unmentioned technical tasks may be considered by those skilled in the art from various embodiments to be described below.

### Technical Solutions

In one technical aspect of the present disclosure, provided is a method of controlling a system including a portable audio output device and a mobile device, the method including executing a meditation-related application through the mobile device, checking a state of a user wearing the portable audio output device connected to the mobile device by wire or wirelessly, outputting a segment related to a first layer of a specific audio content through the portable audio output device depending on the checked state of the user, and based on changing the checked state of the user, outputting a segment related to a second layer of the specific audio content through the portable audio output device, wherein the first layer and the second layer may be different from each other.

The state of the user may include at least one of a brainwave, a heart rate, or a respiration rate.

At least one layer of the specific audio content may include at least one of a layer in a neutral state, a layer in a positive state, or a layer in a negative state.

Based on detecting a frequency of 12 to 30 Hz that is a beta-wave from the brainwave of the user, it may be regarded as the neutral state.

Based on detecting a frequency of 8 to 12 Hz that is an alpha-wave from the brainwave of the user, it may be regarded as the positive state.

Based on detecting a frequency of 30 to 50 Hz that is a gamma-wave from the brainwave of the user, it may be regarded as the negative state.

A length of at least one segment constituting the specific audio content may be the same according to a user and configured in a preset same time unit. Alternatively, a length of at least one segment constituting the specific audio content may be changed depending on a user and may be different according to a profile history of a user-specific state pre-stored in a memory.

Based on the specific audio content related to a narration, a different script may be outputted through the portable audio output device depending on the checked state of the user.

Based on the specific audio content related to music, a brainwave synchronization signal of a different level may be mixed and outputted through the portable audio output apparatus depending on the checked state of the user.

The portable audio output device may include at least one of an earphone, an earbud, or a headset.

In another technical aspect of the present disclosure, provided is a method of controlling a meditation-related application downloadable to a mobile device, the method including controlling to check a state of a user wearing a portable audio output device connected to the mobile device by wire or wirelessly after executing the meditation-related application, controlling to output a segment corresponding to a first layer of a specific audio content through the portable audio output device depending on the checked state of the user, and controlling to output a segment related to a second layer of the specific audio content through the portable audio output device based on changing the checked state of the user, wherein the first layer and the second layer may be different from each other.

### Advantageous Effects

According to an embodiment of the present disclosure, a technology capable of measuring a user's state prior to a full-fledged meditation service is provided.

According to another embodiment of the present disclosure, an algorithm for changing a real-time guide according to a user's changed state is defined even after a meditation service is started (e.g., audio content is output).

In addition, according to further embodiment of the present disclosure, even after a meditation service is terminated, there is a technical effect of providing specific feedback through comparison with past data. Furthermore, it becomes possible to determine a scientific and objective user state, provide feedback, and recommend meditation content more suitable for a user.

The effects obtained from the embodiments are not limited to the effects mentioned above, and other unmentioned effects may be clearly derived and understood by those skilled in the art based on the following detailed description.

### DESCRIPTION OF DRAWINGS

The accompanying drawings, included as part of the detailed description to help the understanding of the embodiments, provide various embodiments and describe the technical features of various embodiments with the detailed description.

FIG. 1 is a diagram for describing an operation of a meditation-related application according to a related art.

FIG. 2 is a diagram illustrating the appearance of a brainwave measurement equipment according to a related art.

FIG. 3 is a diagram illustrating an earbud and a mobile device for measuring a brainwave, a respiratory rate, a heart rate, and the like of a user according to an embodiment of the present disclosure.

FIG. 4 is a flowchart for transmitting and receiving data between a portable audio output device and a mobile device according to an embodiment of the present disclosure.

FIG. 5 is a diagram illustrating an initial operation sequence of checking a user's state by a meditation-related application downloadable to a mobile device or the like according to an embodiment of the present disclosure.

FIG. 6 is a diagram illustrating a process for providing optimized audio content to a user by a meditation-related application downloadable to a mobile device or the like according to an embodiment of the present disclosure.

FIG. 7 is a diagram illustrating content that is changed in real time in order to target user's concentration to a specific level (e.g., 80%) according to an embodiment of the present disclosure.

FIG. 8 is a diagram illustrating a brainwave synchronization signal that is changed in real time in order to target a user's relaxation state to a specific level (e.g., 90%) according to an embodiment of the present disclosure.

FIG. 9 is a diagram schematically illustrating an operation of a system (a portable audio output device, a mobile device, a cloud server, etc.) according to an embodiment of the present disclosure.

FIG. 10 is a diagram illustrating an operation of a meditation-related application according to an embodiment of the present disclosure.

FIG. 11 is a diagram illustrating a process of configuring a segment of image content through a meditation-related application according to an embodiment of the present disclosure in comparison with a related art.

FIG. 12 is a diagram for explaining the relationship between layers and segments of image content in detail through a meditation-related application according to an embodiment of the present disclosure in comparison with a related art.

### BEST MODE

Hereinafter, the embodiments disclosed herein will be described in detail with reference to the accompanying drawings, but components that are the same as or similar to each other regardless of reference numerals will be given the same reference numerals, and redundant description thereof will be omitted. The suffixes “module” and “unit” for components used in the following description are given or used together in consideration of ease of writing the present document, and do not have meanings or roles that are distinct from each other by themselves.

Further, in describing the embodiments disclosed herein, when it is determined that a detailed description of related known technologies may unnecessarily obscure the gist of the embodiments disclosed herein, detailed description thereof will be omitted. In addition, the attached drawings are provided for easy understanding of the embodiments disclosed herein and do not limit technical idea disclosed herein, and should be construed as including all modifications, equivalents, and alternatives falling within the spirit and scope of the present disclosure.

Although terms including ordinal numbers, such as first, second, and the like, may be used to describe various components, the components are not be limited by the terms. The terms are only used to distinguish one component from another. It should be understood that when a component is referred to as being “connected with” another component, the component may be directly connected with another component or an intervening component may also be present.

In contrast, it should be understood that when a component is referred to as being “directly connected with” another component, there is no intervening component present. The terms “includes” or “has” used herein should be understood as not precluding possibility of existence or addition of one or more other characteristics, figures, steps, operations, constituents, components, or combinations thereof.

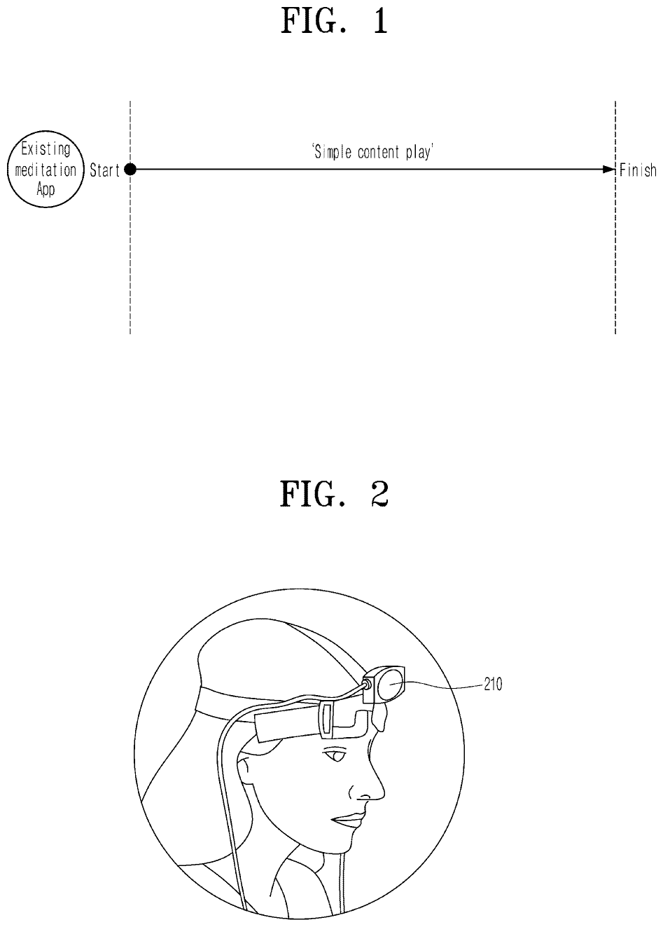

FIG. 1 is a diagram for describing an operation of a meditation-related application according to a related art.

First, referring to FIG. 1, described below is how a meditation-related application according to a related art changes along a time flow.

As shown in FIG. 1, when an existing meditation application is executed, the same content is simply played from a start point (Start) to an end point (Finish), and no change occurs.

That is, there is a problem in that a user does not consider feedback that varies for each user before or even after executing the meditation application.

FIG. 2 shows the appearance of a brainwave measurement equipment according to a related art.

On the other hand, the brainwave measurement equipment shown in FIG. 2 was sometimes used ex post to check the effectiveness and the like of meditation, but it is a form factor that has to be worn on a head, which is burdensome to use in general situations.

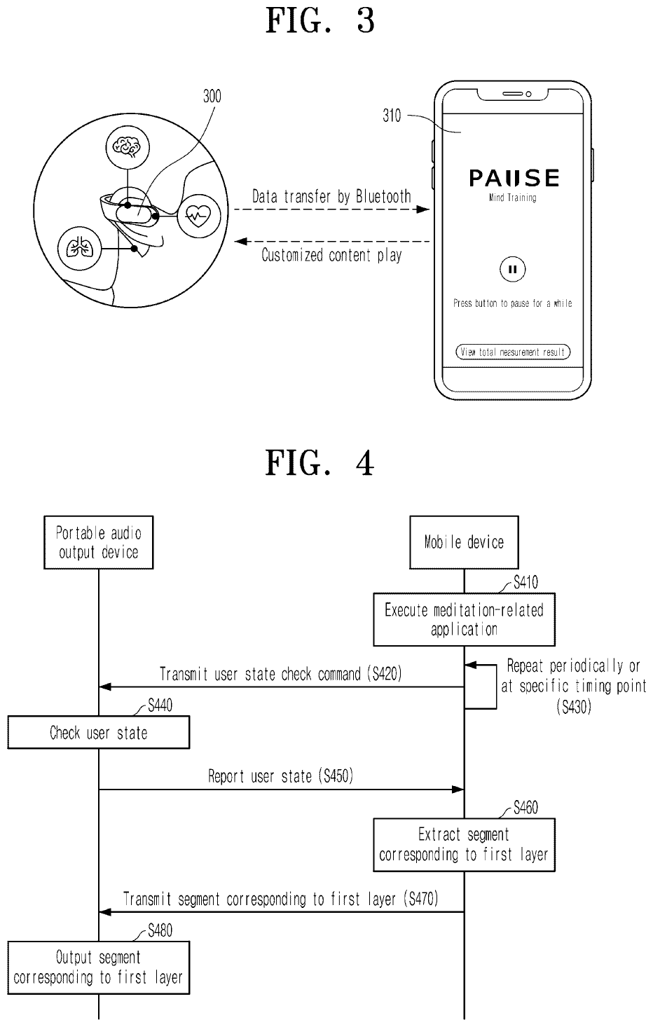

FIG. 3 illustrates an earbud and a mobile device for measuring a user's brain wave, a respiratory rate, a heart rate, and the like according to an embodiment of the present disclosure.

In order to solve the problems of the related art shown in FIG. 1 and FIG. 2, as shown in FIG. 3, an embodiment of the present disclosure proposes a meditation-related application (app.) downloadable to new types of a portable audio output device and a mobile device.

First, a portable audio output device 300 illustrated in FIG. 3 may be, for example, an earphone, an earbud, a headset, or the like, and is designed to be equipped with a function capable of detecting a state of a user wearing the device. Here, the user's state includes at least one of a brainwave, a heart rate, a respiratory rate, etc. for example.

Therefore, the portable audio output device 300 includes, for example, a reference electrode with a constant surface area, a measurement electrode installed apart from the reference electrode to detect and measure a brainwave, a ground electrode separated from the measurement electrode and the reference electrode to be attached to a skin surface, a pressurizing housing pressing the reference electrode, the measurement electrode, and the ground electrode, and a communication module transmitting a brainwave signal measured by the measurement electrode.

Furthermore, the portable audio output device 300 includes, for example, an acceleration sensor module that detects a user's movement as a motion artifact, an infrared sensor module that measures a blood flow amount, a noise removal module that removes the motion artifact from a signal of the measured blood flow amount, and a heart rate calculation module that calculates a heart rate based on the blood flow amount signal from which the motion artifact has been removed.

In addition, the portable audio output device 300 further includes, for example, a respiratory rate measurement module that detects a respiratory signal for a user's respiration with a gyro sensor and measures a respiratory rate from the detected respiratory signal.

Yet, the modules described above are only examples, and it will be said that the adoption of other components capable of checking a brainwave, a heart rate, or a respiratory rats falls within the scope of other rights of the present disclosure.

Referring back to FIG. 3, it is assumed that a meditation-related application is downloaded to a mobile device 310 according to an embodiment of the present disclosure.

The mobile device 310 is communicatively connected to the portable audio output device 300 by wire or wirelessly, and transmits and receives data through, for example, Bluetooth (BT) wireless communication.

The mobile device 310 is designed to transmit customized content back to the portable audio output device 300, based on the user's state information received from the portable audio output device 300.

Specific data transmitted and received between the portable audio output device 300 and the mobile device 310 will be described in more detail with reference to FIG. 4.

FIG. 4 is a flowchart for transmitting and receiving data between a portable audio output device and a mobile device according to an embodiment of the present disclosure.

As shown in FIG. 4, a system according to an embodiment of the present disclosure includes a portable audio output device and a mobile device, but it is also possible to implement the present disclosure for each device.

The mobile device executes a meditation-related application (S410).

Furthermore, the mobile device transmits a command for checking a user state to the portable audio output device connected by wire or wirelessly (S420). However, the command transmission (S420) is repeated at regular intervals or at a specific timing point (S430).

Then, the portable audio output device checks a user's state (S440) and reports the user's state to the mobile device (S450). Here, the user's state includes, for example, at least one of a brainwave, a heart rate, or a respiratory rate.

The mobile device is designed to extract a segment corresponding to a first layer of a specific audio content according to the user's state reported by the portable audio output device (S460).

The mobile device transmits the segment corresponding to the first layer to the portable audio output device (S470), and the portable audio output device outputs the segment corresponding to the first layer (S480).

Furthermore, as described above, when the user's state checked through the step S430 is changed, a segment corresponding to a second layer of the specific audio content is outputted through the portable audio output device.

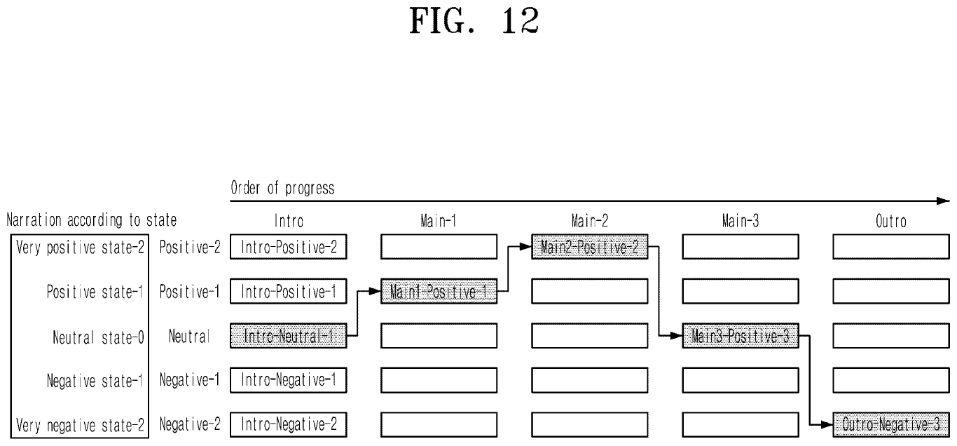

In particular, the first layer and the second layer are characterized in being different from each other, and the layer and segment, which is one of the features of the present disclosure, will be described in more detail with reference to FIG. 12.

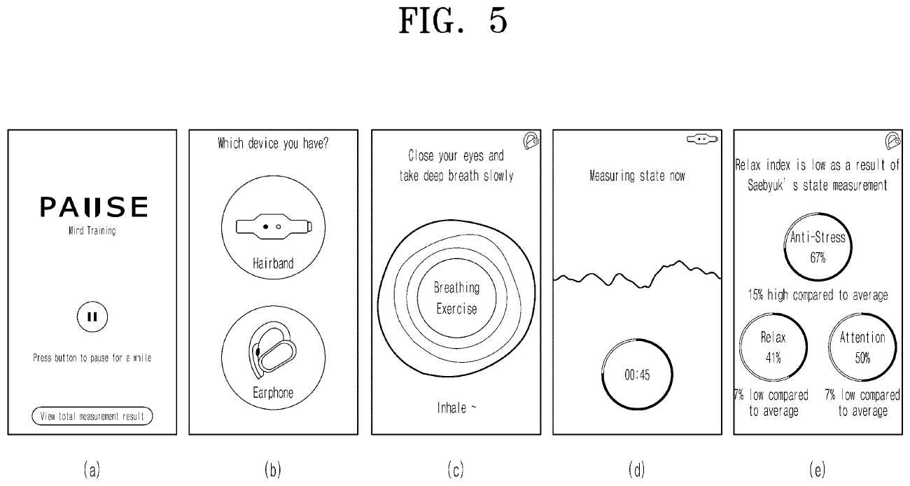

FIG. 5 illustrates an initial operation sequence in which a meditation-related application downloadable to a mobile device or the like checks a user's state according to an embodiment of the present disclosure.

As described above, according to one of the features of the present disclosure, it is designed to check a user's state without playing an audio content right after executing a meditation-related application.

For example, as shown in FIG. 5(a), when a meditation-related application downloaded on a mobile device is executed, an initial guide screen is displayed.

When a button located in the center shown in FIG. 5(a) is pressed, as shown in FIG. 5(b), a GUI is displayed to ask if there is a device that is able to check a user's state while attached to a user's body.

When a portable audio output device (e.g., an earphone) is selected from the devices shown in FIG. 5(b), as shown in FIG. 5(c), a guide message for a user to be stable is displayed.

After the guide message shown in FIG. 5(c) is displayed for a predetermined time (T1), as shown in FIG. 5(d), a second guide message indicating that the user's state is being measured is displayed.

After the guide message shown in FIG. 5(d) is displayed for a predetermined time (T2 greater than T1), as shown in FIG. 5(e), a user's state measurement result is displayed. Furthermore, as shown in FIG. 5(e), the user's state measurement result is displayed by comparing and showing a numerical value of the corresponding user with respect to the average, thereby being advantageous in that the user can receive feedback on a relative state of his or her own.

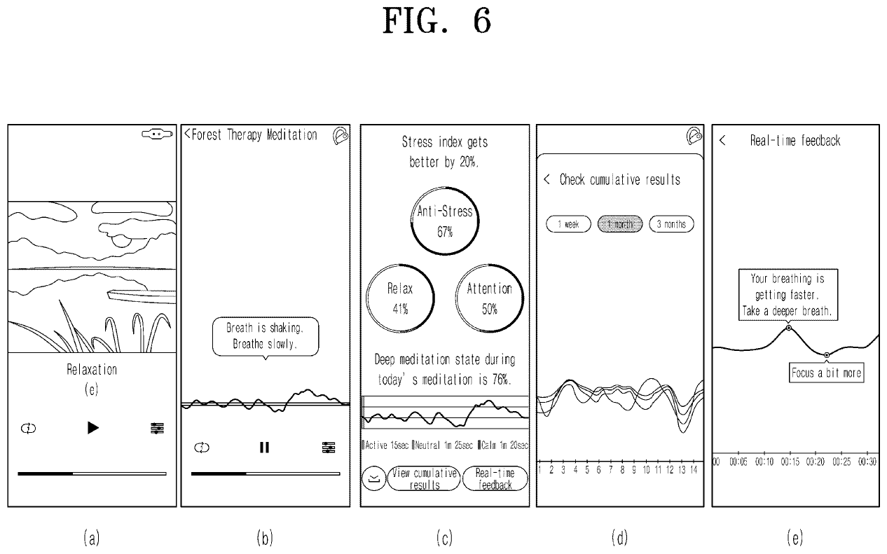

FIG. 6 illustrates a process in which a meditation-related application downloadable to a mobile device or the like provides optimized audio content and the like to a user according to an embodiment of the present disclosure.

When all the user state checks illustrated in FIG. 5 are completed, as illustrated in FIG. 6, customized audio for a current state of the user is outputted.

First, as shown in FIG. 6(a), an optimal audio content is selected according to the user's state (e.g., brainwave, heart rate, and respiratory rate measurement results are considered) and outputted through a portable audio output device.

Furthermore, unlike the related art, as shown in FIG. 6(b), the user state check is maintained continuously or periodically even after a random audio content is outputted. Therefore, when the user's breathing is measured to be shaking, as shown in FIG. 6(b), a guide message “Breathing is shaking. Take your time and take a deep breath” is designed to be outputted through the mobile device or a voice through the portable audio output device.

In addition, as shown in FIG. 6(c), after the play of the meditation-related audio content is completed, it is designed to graphically display the result value of the user's state change.

Furthermore, as shown in the lower part of FIG. 6(c), it is designed to additionally display a first option to view cumulative results and a second option to check real-time feedback.

When the first option (view cumulative result) shown in the lower end of FIG. 6(c) is selected, as shown in FIG. 6(d), a graph capable of showing a result of a past meditation experience of the corresponding user is displayed. The cumulative result, as shown in FIG. 6(d), may be selected in units of one week, one month, and three months, or may be designed in other units.

On the other hand, when the second option (real-time feedback) shown in the lower end of FIG. 6(c) is selected, the moment when a specific message occurs during the meditation experience that has just been made (i.e., the point where the user state change has occurred rapidly) is highlighted and displayed.

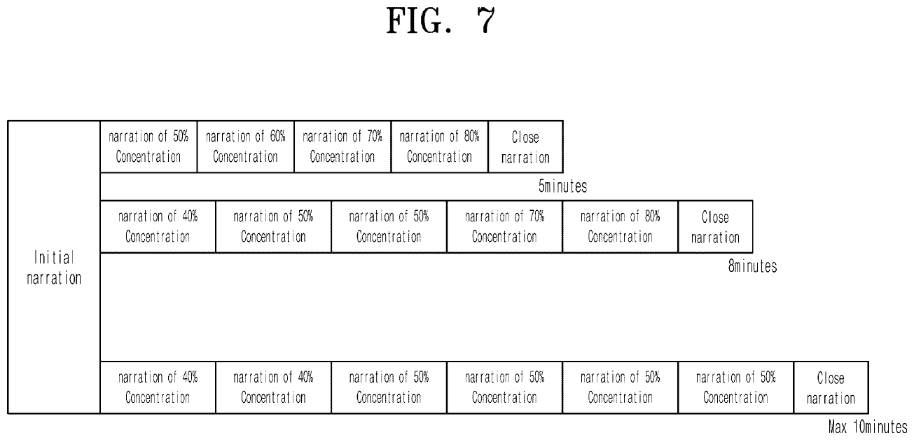

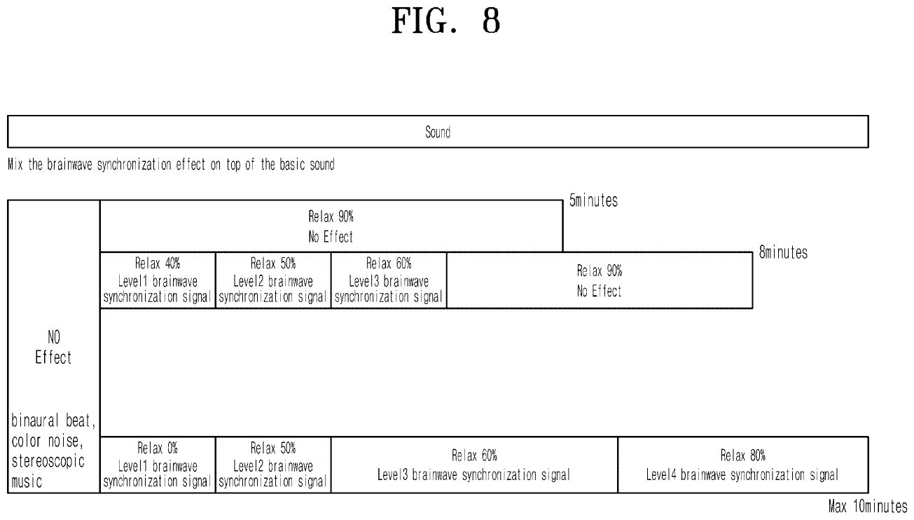

FIG. 7 shows a content that is changed in real time in order to target user's concentration to a specific level (e.g., 80%) according to an embodiment of the present disclosure.

According to an embodiment of the present disclosure, a length of an audio content is designed to vary depending on user's immersion in meditation.

Therefore, a time (i.e., total length) of a time for ending meditation is designed to vary for each user. For example, for a user with high meditation immersion, meditation is designed to end shorter. On the contrary, for a user with low meditation immersion, meditation is designed to end relatively longer.

However, even if the immersion of meditation is low, it is a waste of resources to output audio content forever, so a max limit time of audio content is set so that it can be terminated even if a desired concentration is not reached.

FIG. 7 assumes a case in which meditation content, for which concentration is set to 80%, is played.

First of all, it is assumed that user's concentration reaches 80% of a target value within a relatively short time (5 minutes) based on the graph shown at a top end of FIG. 7.

If user's concentration is determined as 50%, a corresponding narration is outputted through a portable audio output device. If user's concentration is determined as 40%, a corresponding narration is outputted through the portable audio output device. If user's concentration is determined as 70%, a corresponding narration is outputted through the portable audio output device. If user's concentration is determined as 80%, a corresponding narration is outputted through the portable audio output device and a close narration is outputted.

On the other hand, when describing based on a graph shown in the middle of FIG. 7, it is assumed that user's concentration reaches 80% that is a target value within a relatively long time (8 minutes).

If user's concentration is determined as 40%, a corresponding narration is outputted through the portable audio output device. If user's concentration is determined as 50%, a corresponding narration is outputted through the portable audio output device. If user's concentration is determined as 70%, a corresponding narration is outputted through the portable audio output device, If user's concentration is determined as 80%, a corresponding narration is outputted through the portable audio output device and a close narration is outputted.

Finally, based on the graph shown at a bottom end of FIG. 7, it is assumed that user's concentration fails to reach 80% that is a target value within 10 minutes in which user's concentration is maximum (Max).

After the meditation-related audio content is outputted, if the concentration 80% that is a target value is not reached within a preset time limit of 10 minutes, it is outputted through the device, and a close narration is outputted.

As described above in FIG. 7, when the meditation-related audio content corresponds to a narration, another script is controlled to be outputted through the portable audio output device according to a user's checked state.

FIG. 8 shows a brainwave synchronization signal that is changed in real time in order to target a user's relaxation state to a specific level (e.g., 90%) according to an embodiment of the present disclosure.

First, it is designed to output a brainwave synchronization effect by mixing it on a meditation-related audio sound.

Based on the graph shown at the top end of FIG. 8, it is assumed that a user's relaxation state reaches 90% that is a target value within a relatively short time (5 minutes).

Based on the graph shown in the middle of FIG. 8, it is assumed that a user's relaxation state reaches 90% that is a target value within a relatively late time (8 minutes).

If a user's relaxation state is determined to be 40%, a brainwave synchronization signal of level 1 corresponding thereto is mixed and outputted through a portable audio output device. If a user's relaxation state is determined to be 50%, a brainwave synchronization signal of level 2 corresponding thereto is mixed and outputted through the portable audio output device. If a user's relaxation state is determined to be 60%, a brainwave synchronization signal of level 3 corresponding thereto is mixed and outputted. If a user's relaxation state is determined to be 90%, it is designed to output only an original sound without mixing a brainwave synchronization signal anymore.

Finally, based on the graph shown at the bottom end of FIG. 8, it is assumed that a user's relaxation state fails to reach 90% that is a target value within 10 minutes that is the time in which a user's relaxation state is maximum.

If a user's relaxation state is determined to be 40%, a brainwave synchronization signal of level 1 corresponding thereto is mixed and outputted through the portable audio output device. If a user's relaxation state is determined to be 50%, a brainwave synchronization signal of level 2 corresponding thereto is mixed and outputted through the portable audio output device. If a user's relaxation state is determined to be 60%, a brainwave synchronization signal of level 3 corresponding thereto is mixed and outputted. If a user's relaxation state is determined to be 80%, a brainwave synchronization signal of level 4corresponding thereto is mixed and outputted. However, if a target value of relaxation 90% is not reached within 10 minutes, which is a preset time limit, after the meditation-related audio content is outputted, an output of the audio content is stopped to reduce waste of resources and time.

As described above in FIG. 8, when the meditation-related audio content corresponds to music, a brainwave synchronization signal of a different level is controlled to be mixed and outputted through the portable audio output device according to a checked user's state.

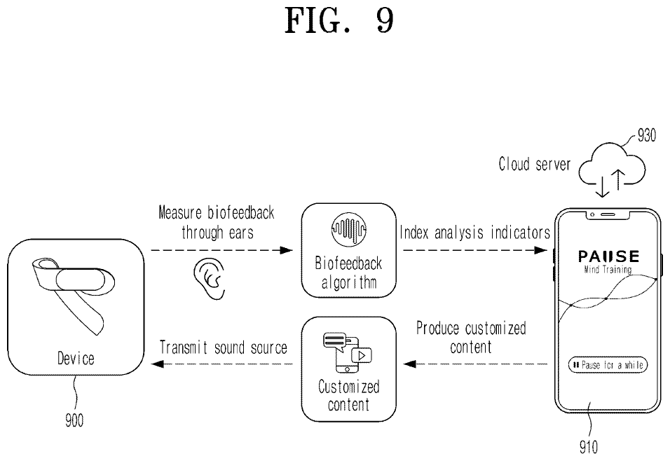

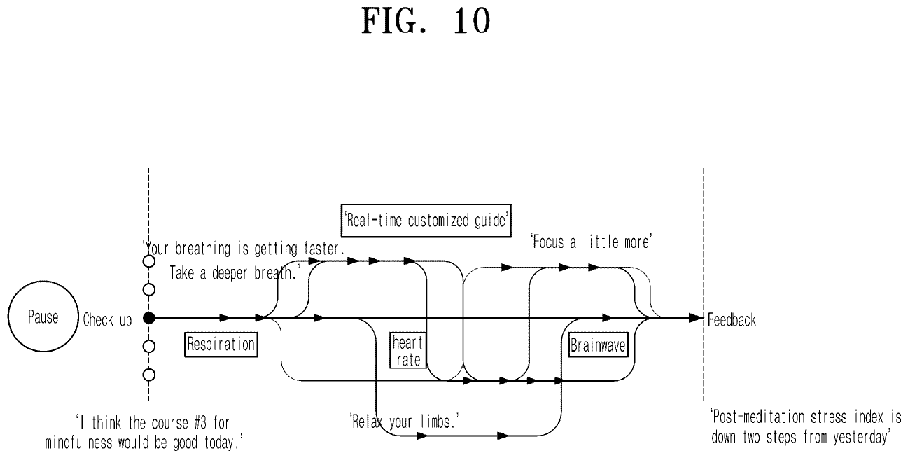

FIG. 9 is a diagram schematically illustrating an operation of a system (e.g., portable audio output device, mobile device, cloud server, etc.) according to an embodiment of the present disclosure.

Unlike the system illustrated in FIG. 3, a system illustrated in FIG. 9 additionally includes a cloud server.

When a meditation-related application downloaded to a mobile device 910 is executed, a short-range wired or wireless communication connection with a portable audio output device 900 is established. Furthermore, the mobile device 910 may additionally receive state-related information of other users and the like through remote communication with a cloud server 930.

When the portable audio output device 900 is, for example, an earphone/earbud, or the like, the mobile device 910 measures the biofeedback through a user's ear and indexes an analysis index.

In addition, the mobile device 910 is designed to receive a customized content according to a user state from the cloud server 930 and transmit it in real time to the portable audio output device 900.

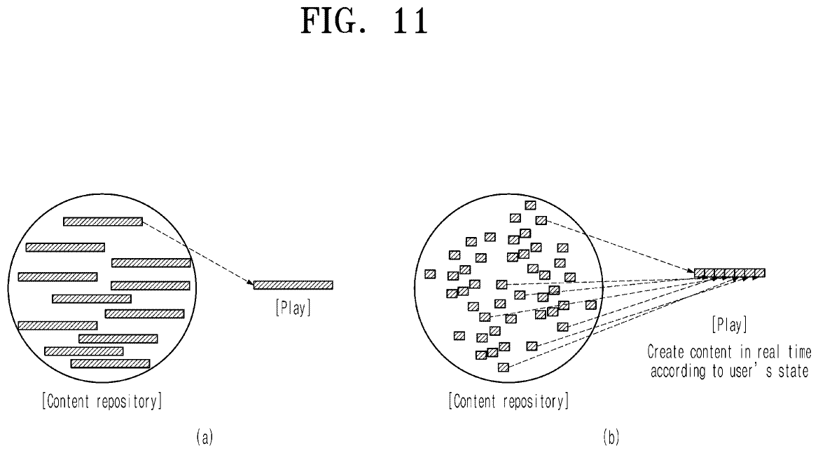

FIG. 10 is a diagram illustrating an operation of a meditation-related application according to an embodiment of the present disclosure.

A name of a meditation-related application according to an embodiment of the present disclosure may be named as ‘pause’, but this is only an example, and the present disclosure is not limited to the corresponding name.

Unlike the related art, when a meditation-related application according to an embodiment of the present disclosure is executed, it is designed to check user's current states (e.g., brainwaves, heart rate, respiratory rate, etc.) from various angles through a portable audio output device or the like.

In addition, according to the checked user's current state, a mobile device transmits an audio content (e.g., mindfulness course #3) optimized for the user's current state to the portable audio output device.

Furthermore, even after the portable audio output device outputs the corresponding audio content, the user's state is continuously monitored periodically or in real time.

For example, when a third of a meditation-related audio content is played, if a user has a breathing problem in the user's state, a message ‘Your breathing is getting faster. Take a deeper breath.’ is displayed through the mobile device or outputted as audio through the portable audio output device.

For example, when two-thirds of the meditation-related audio content is played, if the user has a heart rate problem in the user's state, a message ‘Relax your limbs.’ is displayed on the mobile device or outputted as audio through the portable audio output device.

For example, just before the end of the meditation-related audio content, if there is a problem with brainwaves in the user's state, a message “Focus a little more” is displayed on the mobile device or outputted as audio through the portable audio output device.

Additionally, when the meditation-related audio content is finished, a message of comparison with past state information (e.g., post-meditation stress index is down two steps from yesterday) is displayed through the mobile device or outputted as audio through the portable audio output device.

That is, unlike the related art, the present disclosure should continuously check a state from the time a user executes a meditation-related application to the end and provide a completely different type of meditation-related audio. To implement this, a specific technique for segmenting an audio content by layer will be described in more detail with reference to FIG. 11 and FIG. 12.

FIG. 11 is a diagram illustrating a process of configuring a segment of an image content through a meditation-related application according to an embodiment of the present disclosure in comparison with the related art.

According to the related art, as shown in FIG. 11(a), meditation-related content stored in a server (e.g., a content repository) is being played until the end of one song on a mobile device and a portable audio device. In other words, a user state is not considered at all.

On the other hand, according to an embodiment of the present disclosure, as shown in FIG. 11(b), all individual meditation-related contents are designed to be segmented into very small units to generate a content according to a user's state in real time.

In order to implement the corresponding technology, it is necessary to create an algorithm that divides a content into small units and connects the content according to a user's state. A relevant detailed embodiment will be described in more detail with reference to FIG. 12.

In addition, FIG. 12 is a diagram for describing the relationship between a layer and a segment of an image content in detail through a meditation-related application according to an embodiment of the present disclosure in comparison with the related art.

As shown in FIG. 12, a meditation-related application according to an embodiment of the present disclosure is designed to change a layer of a prepared narration according to the degree of immersion of meditation. On the other hand, a user state, such as the degree of immersion in meditation or the like, is extracted from biofeedback (e.g., brainwaves, heart rate, breathing, etc.) through a portable audio output device as described above.

More specifically, at least one layer of the meditation-related audio content includes at least one of a layer in a neutral state, a layer in a positive state, and a layer in a negative state.

Furthermore, for example, it is designed to be considered a neutral state if a frequency of 12 to 30 Hz, which is a beta wave, is detected from in user's brainwaves, a positive state if a frequency of 8 to 12 Hz, which is an alpha a wave, is detected from the user's brainwaves, and a negative state if a frequency of 30 to 50 Hz, which is a gamma wave, is detected from the user's brainwaves. Of course, it is also within the scope of other rights of the present disclosure to subdivide a user's state and distinguish layers of a content based on other parameters (e.g., heart rate, respiratory rate, etc.).

In addition, as shown in FIG. 12, dividing the negative state into two levels (e.g., negative state and very negative state) and dividing the positive state into two levels (e.g., very positive state and positive state) is also within the scope of other rights of the present disclosure.

Furthermore, as shown in FIG. 12, a length of at least one segment (Intro, Main-1, Main-2, Main-3, and Outro) constituting a specific audio content is the same without being changed depending on a user, and may be fixed in a preset same time unit (e.g., 1 minute, 2 minutes, etc.).

Yet, when referring to a profile history stored in the memory, for example, while watching 10 minutes of meditation-related audio, there may be a user who has a user-specific state change frequently occurring, and conversely, there may be a user who has a user-specific state change occurring relatively less frequently.

In consideration of this, according to another embodiment of the present disclosure, a length of a segment constituting a meditation-related audio content is designed to change depending on a user, and in particular, it is designed to vary according to a profile history of a user-specific state previously stored in a memory.

More specifically, for example, when a first user whose state change frequently occurs executes a meditation-related application, a segment (e.g., 1-minute unit) is designed to be more densely split. When designed in this way, there is a technical effect that may provide a meditation-related content that is more optimized for a state change of a sensitive user.

On the other hand, when a second user, whose state change occurs relatively less frequently, executes a meditation-related application, it is designed to divide a segment (e.g., 2-minutes and 30-seconds unit) into a wider period. When designed in this way, there is an advantage of minimizing unnecessary user state check.

Furthermore, when a user's state shown in FIG. 12 is a neutral state (neutral state), a narration (e.g., focusing well) of a corresponding layer is outputted through a portable audio output device.

On the other hand, if a user's state is positive, a narration of a corresponding layer (e.g., focusing very well) is outputted through the portable audio output device, or the corresponding narration is controlled not to be outputted.

In addition, if a user's state is negative, a narration of a corresponding layer (e.g., please focus on meditation) is outputted through the portable audio output device.

Namely, even if the same meditation audio is selected, there is a technical effect that an optimal meditation can be provided for each user and according to a timing point (state) even for the same user.

On the other hand, according to the related art, meditation-related audio is not divided into segments, and the concept of a layer does not exist, as shown in FIG. 12. For example, the same audio assuming a neutral state is outputted unchanged.

According to an embodiment of the present disclosure, a customized meditation service is provided by changing a content in real time through analysis of body data (i.e., biofeedback: brainwaves, heart rate, breathing, etc.). As meditation-related audio, popular music, ASMR, guided meditation, and audio books may be used, and an appropriate content is applied according to each state of body data.

As described above, the meditation-related application according to the related art does not automatically determine my-state before meditation, but has a problem that it costs a lot of money and time because a user has to find a meditation audio individually desired. However, a meditation-related service according to an embodiment of the present disclosure has the advantage of automatically finding appropriate meditation content by checking a user's state.

Furthermore, the meditation-related application according to the related art provided the same message as to whether meditation is well performed only in a one-sided delivery way during meditation. However, a meditation-related service according to an embodiment of the present disclosure is designed to continuously monitor a user's state even after a related audio is outputted and continuously provide the result (“Breathe deeper”, etc.).

In addition, the meditation-related application according to the related art did not provide feedback after meditation. However, a meditation-related service according to an embodiment of the present disclosure has the advantage of providing feedback (e.g., “You're doing well, but you need to concentrate more”) on how much meditation-related improvement has been achieved by referring to previous data.

The various embodiments of the present invention have been described above, but these are merely examples, and the present invention is not limited thereto. The invention should be construed as having the broadest scope in accordance with the basic principles disclosed in this specification.

Those skilled in the art may combine and substitute the disclosed embodiments to implement embodiments not explicitly described, but such implementations are also within the scope of the present invention. Furthermore, those skilled in the art can easily make modifications or alterations to the disclosed embodiments based on this specification, and such modifications or alterations are clearly within the scope of the present invention.

### MODE FOR DISCLOSURE

In the best form for the implementation of the present disclosure, which is the previous table of contents, various forms for implementing the present disclosure have been described above.

### INDUSTRIAL APPLICABILITY

Since an embodiment of the present disclosure is applicable to a portable audio output device, a mobile device, a cloud server, a meditation-related application, and the like, industrial applicability is recognized.

What is claimed is:

1. A method of controlling a system including a portable audio output device and a mobile device, the method comprising:
executing a meditation-related application through the mobile device;
checking a state of a user wearing the portable audio output device connected to the mobile device by wire or wirelessly;
outputting a segment related to a first layer of a specific audio content through the portable audio output device depending on the checked state of the user; and
based on changing the checked state of the user, outputting a segment related to a second layer of the specific audio content through the portable audio output device,
wherein the first layer and the second layer are different from each other.

2. The method of claim 1, wherein the state of the user includes at least one of a brainwave, a heart rate, or a respiration rate.

3. The method of claim 2, wherein at least one layer of the specific audio content comprises at least one of a layer in a neutral state, a layer in a positive state, or a layer in a negative state.

4. The method of claim 3, wherein based on detecting a frequency of 12 to 30 Hz that is a beta-wave from the brainwave of the user, it is regarded as the neutral state, wherein based on detecting a frequency of 8 to 12 Hz that is an alpha-wave from the brainwave of the user, it is regarded as the positive state, and wherein based on detecting a frequency of 30 to 50 Hz that is a gamma-wave from the brainwave of the user, it is regarded as the negative state.

5. The method of claim 3, wherein a length of at least one segment constituting the specific audio content is the same according to a user and configured in a preset same time unit.

6. The method of claim 3, wherein a length of at least one segment constituting the specific audio content is changed depending on a user and is different according to a profile history of a user-specific state pre-stored in a memory.

7. The method of claim 1, further comprising based on the specific audio content related to a narration, outputting a different script through the portable audio output device depending on the checked state of the user.

8. The method of claim 1, further comprising based on the specific audio content related to music, mixing and outputting a brainwave synchronization signal of a different level through the portable audio output device depending on the checked state of the user.

9. The method of claim 1, wherein the portable audio output device comprises at least one of an earphone, an earbud, or a headset.

10. A method of controlling a meditation-related application downloadable to a mobile device, the method comprising:
controlling to check a state of a user wearing a portable audio output device connected to the mobile device by wire or wirelessly after executing the meditation-related application;
controlling to output a segment corresponding to a first layer of a specific audio content through the portable audio output device depending on the checked state of the user; and
controlling to output a segment related to a second layer of the specific audio content through the portable audio output device based on changing the checked state of the user,
wherein the first layer and the second layer are different from each other.
